(19) United States
(12) Patent Application Publication
(10) Pub. No.: US 2023/0377114 A1
(43) Pub. Date: Nov. 23, 2023

(54) SYSTEMS AND METHODS FOR DIAGNOSING OPERATIONAL ISSUES IN A RESPIRATORY SYSTEM

(71) Applicant: ResMed Sensor Technologies Limited, Dublin (IE)

(72) Inventors: Redmond Shouldice, Dublin (IE); Stephen McMahon, Dublin (IE)

(21) Appl. No.: 18/028,337

(22) PCT Filed: Sep. 24, 2021
(86) PCT No.: PCT/IB2021/058744

§ 371 Date: Mar. 24, 2023

Related U.S. Application Data
(60) Provisional application No. 63/083,525, filed on Sep. 25, 2020.

(51) Int. Cl. G06T 7/00; A61M 16/00

(52) U.S. Cl. CPC G06T 7/0002; A61M 16/0003; A61M 2205/3375; G06T 2207/30

(57) ABSTRACT

The present disclosure relates to a method for diagnosing an operational issue in a respiratory therapy system. A command is received, via an external device, to begin diagnosing the operational issue in the respiratory therapy system. One or more sensors of the external device are caused to generate acoustic data, which is indicative of one or more sounds emanating from the respiratory therapy system. At least a portion of the generated acoustic data is analyzed to identify (i) a location of the operational issue in the respiratory therapy system, (ii) one or more causes of the operational issue in the respiratory therapy system, or (iii) both (i) and (ii).

### CROSS-REFERENCE TO RELATED APPLICATIONS

This application claims the benefit of, and priority to, U.S. Provisional Patent Application No. 63/083,525 filed on Sep. 25, 2020, which is hereby incorporated by reference herein in its entirety.

### TECHNICAL FIELD

The present disclosure relates generally to systems and methods for diagnosing operational issues in a mechanical device, and more particularly, to systems and methods for diagnosing operational issues in a respiratory therapy system.

### BACKGROUND

Various systems exist for aiding users experiencing sleep apnea and related respiratory disorders. A range of respiratory disorders exist that can impact users. Certain disorders are characterized by particular events (e.g., apneas, hypopneas, hyperpneas, or any combination thereof). Examples of respiratory disorders include periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders. One or more components within a respiratory therapy system may have operational issues that can lead to a user complaint and return, or cause the user to reject respiratory therapy. Thus, a need exists for systems and methods for diagnosing operational issues in the respiratory therapy system. The present disclosure is directed to solving these and other problems.

### SUMMARY

According to some implementations of the present disclosure, a method for diagnosing an operational issue in a respiratory therapy system is disclosed. A command is received, via an external device, to begin diagnosing the operational issue in the respiratory therapy system. One or more sensors of the external device are caused to generate acoustic data, which is indicative of one or more sounds emanating from the respiratory therapy system. At least a portion of the generated acoustic data is analyzed to identify (i) a location of the operational issue in the respiratory therapy system, (ii) one or more causes of the operational issue in the respiratory therapy system, or (iii) both (i) and (ii).

analyzing at least a portion of the generated acoustic data to identify (i) a location of the operational issue in the respiratory therapy system, (ii) one or more causes of the operational issue in the respiratory therapy system, or (iii) both (i) and (ii).

According to some implementations of the present disclosure, a system includes a control system having one or more processors, and a memory having stored thereon machine readable instructions. The control system is coupled to the memory. Any of the methods disclosed above, and further described herein, is implemented when the machine executable instructions in the memory are executed by at least one of the one or more processors of the control system.

According to some implementations of the present disclosure, a system for diagnosing an operational issue in a respiratory therapy system includes a control system having one or more processors configured to implement any of the methods disclosed above and further described herein.

According to some implementations of the present disclosure, a computer program product includes instructions which, when executed by a computer, cause the computer to carry out any of the methods disclosed above and further described herein. In some implementations, the computer program product is a non-transitory computer readable medium.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system, according to some implementations of the present disclosure.

FIG. 2 is a perspective view of at least a portion of the system of FIG. 1, a user wearing a full face mask, and a bed partner, according to some implementations of the present disclosure.

FIG. 3 is a flow diagram for a method for diagnosing an operational issue in a respiratory therapy system, according to some implementations of the present disclosure.

FIG. 4 illustrates the generation of acoustic data in response to an acoustic reflection associated with a user while using a respiratory therapy system, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hyper tension, diabetes, stroke, insomnia, and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

In a conventional respiratory therapy system, when one or more components of the respiratory therapy system experience operational issues, the diagnostics is reactive, which requires the operational issues to be so problematic that the user complains to their medical team or the manufacturer of the respiratory therapy system. These conventional methods can be slow to realize these field issues with the components. In addition, some users may never complain, and simply stop using the respiratory therapy system or move to another therapy system. Thus, there exists a need for systems and methods for diagnosing operational issues in a respiratory therapy system, such as the issues disclosed herein, so that the respiratory therapy system can function properly to aid the user in achieving desired therapy results, which in turn improves long-term user adherence. In addition, when the operational issues are diagnosed early and/or accurately, unnecessary shipment costs, returns, and lab time (e.g., for disinfection) can be reduced.

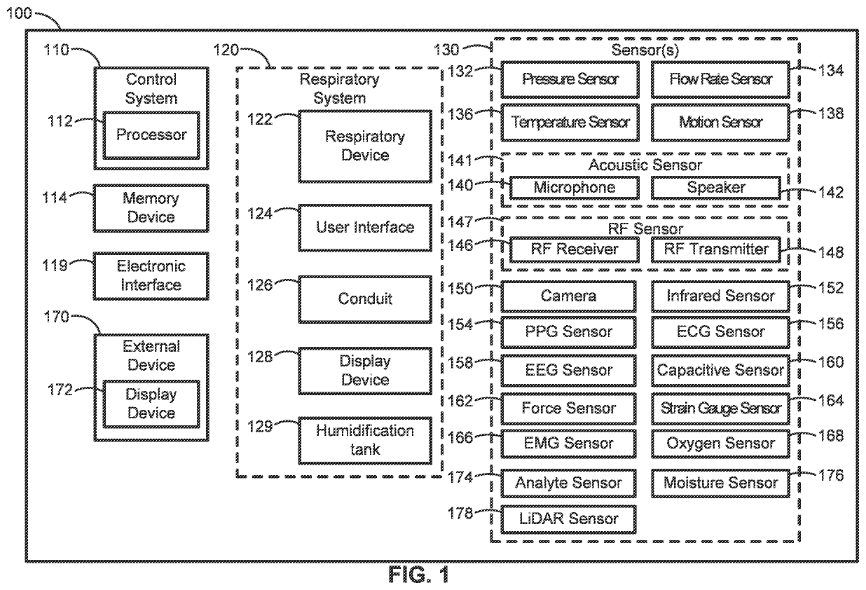

Referring to FIG. 1, a system 100, according to some implementations of the present disclosure, is illustrated. The system 100 is for providing a variety of different sensors related to a user's use of a respiratory therapy system, among other uses. The system 100 includes a control system 110, a memory device 114, an electronic interface 119, one or more sensors 130, and one or more external devices/user devices 170. In some implementation, the system 100 further includes a respiratory system 120 (also called “respiratory therapy system”) that includes a respiratory therapy device 122. The system 100 can be used to diagnose an operational issue in a respiratory therapy system, as disclosed in further detail herein.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia, an employment status of the user, an educational status of the user, a socioeconomic status of the user, or any combination thereof. The medical information can include, for example, including indicative of one or more medical conditions associated with the user, medication usage by the user, or both. The medical information can also include a fall risk assessment associated with the user (e.g., a fall risk score using the Morse fall scale). The medical information data can further include a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof. In some implementations, the memory device 114 stores media content that can be displayed on the display device 128.

The electronic interface 119 is configured to receive data (e.g., physiological data) from the one or more sensors 130 such that the data can be stored in the memory device 114 and/or analyzed by the processor 112 of the control system 110. In some implementations, the external device 170 includes the electronic interface 119, or another electronic interface that is the same as the electronic interface 119 described herein. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

In some implementations, the external device 170 (e.g., a smartphone as implemented in certain examples in this disclosure) can be in communication with the respiratory therapy device 122 via a wired connection or a wireless connection (e.g., using an RF communication protocol, a WiFi communication protocol, a Bluetooth communication protocol, an IR communication protocol, over a cellular network, over any other optical communication protocol, etc.). As such, the external device 170 can run a test to detect potential operational issues. For example, the external device 170 may be configured to ramp the speed of the motor in the respiratory therapy device 122 while generating acoustic data, and thereby potentially amplifying any operational issues that are more obvious at higher motor speeds (or different motor speeds). In some such examples, the test is run when the user interface 124 is not on the face of the user, because the higher air pressure (caused by the ramping of the motor) may be uncomfortable for the user. Running of the test when the user interface 124 is not on the face of the user advantageously allows testing involving motor speeds and air pressures, which cannot be tolerated by the user if the user interface 124 were on the face of the user. In some other such examples, a similar test could be carried out during the normal ramping of air pressure during a therapy session (e.g., from a nominal initial pressure such as 4 cmH₂O to a prescribed therapy pressure such as 10 cmH₂O), where the increasing/increased air pressure is tolerated by the user.

The respiratory therapy system 120 can include a respiratory pressure therapy device (referred to as the respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

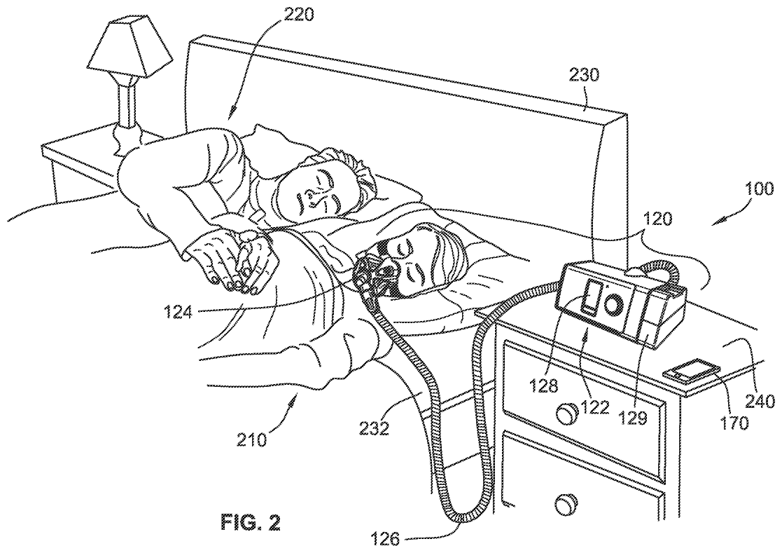

As shown in FIG. 2, in some implementations, the user interface 124 is or includes a facial mask that covers the nose and mouth of the user. Alternatively, in some implementations, the user interface 124 is a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 comprises a mouthpiece (e.g., a night guard mouthpiece molded to conform to the user's teeth, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score such as a myAir™ score, such as described in WO 2016/061629 and U.S. Patent Pub. No. 2017/0311879, which are hereby incorporated by reference herein in their entireties; the current date/time; personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

In some implementations, the system 100 can be used to deliver at least a portion of a substance from the receptacle 180 to the air pathway the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Still referring to FIG. 1, the one or more sensors 130 of the system 100 include a pressure sensor 132, a flow rate sensor 134, a temperature sensor 136, a motion sensor 138, the microphone 140, the speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the IR sensor 152, the PPG sensor 154, the ECG sensor 156, the EEG sensor 158, the capacitive sensor 160, the force sensor 162, the strain gauge sensor 164, the EMG sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

In some implementations, the physiological data generated by one or more of the sensors 130 can be used by the control system 110 to determine a sleep-wake signal associated with a user during the sleep session and one or more sleep-related parameters. The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, or any combination thereof. The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof.

Generally, the sleep session includes any point in time after the user 210 has laid or sat down in the bed 230 (or another area or object on which they intend to sleep), and has turned on the respiratory therapy device 122 and donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory therapy device 122, and gets out of bed 230. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user 210, ending when the respiratory therapy device 122 stops supplying the pressurized air to the airway of the user 210, and including some or all of the time points in between, when the user 210 is asleep or awake.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122, the user interface 124, or the conduit 126. The pressure sensor 132 can be used to determine an air pressure in the respiratory therapy device 122, an air pressure in the conduit 126, an air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. Examples of flow rate sensors (such as, for example, the flow rate sensor 134) are described in International Publication No. WO 2012/012835 and U.S. Pat. No. 10,328,219, both of which are hereby incorporated by reference herein in their entireties. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperature data indicative of a core body temperature of the user 210 (FIG. 2), a skin temperature of the user 210, a temperature of the air flowing from the respiratory therapy device 122 and/or through the conduit 126, a temperature of the air in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

In some implementations, the motion sensor 138 is external to the respiratory therapy device 122 and/or the respiratory therapy system 120. For example, the motion sensor 138 may be an accelerometer comprised in the external device 170 such as a smart device (e.g., a smartphone), which can be used to detect aberrant vibrations from the respiratory therapy device 122 by being placed on or near the respiratory therapy device 122 (e.g., near the motor, when motor is running, at one or more speeds, and/or during a ramp in speed of the motor). In some such implementations, the vibration data generated by the motion sensor 138 can be compared to a library of normal and/or expected aberrant vibrations (e.g., a library of vibration data including, for example, vibration frequencies), or features extracted from vibration data (and/or the raw vibration data itself) can be inputted to trained machine learning model, wherein the model is trained using vibration data (and/or features extracted therefrom) from respiratory therapy devices and information relating to corresponding operational issues of the respiratory therapy devices. This vibration data and analysis thereof can be used alone, and/or to complement (e.g., to confirm) the analyses performed using the acoustic data generated by the microphone 140, as described herein.

The microphone 140 may be coupled to or integrated in an external device (e.g., the user device 170) and outputs sound data that can be stored in the memory device 114 and/or analyzed by the processor 112 of the control system 110. For example, in some implementations, the microphone 140 can be integrated in and/or coupled to a co-located smart device, such as the user device 170, a TV, a watch (e.g., a mechanical watch or the smart device 270), a pendant, the mattress 232 (FIG. 2), the bed 230 (FIG. 2), beddings positioned on the bed 230 (FIG. 2), the pillow, a speaker (e.g., the speaker 142 of FIG. 1), a radio, a tablet, a waterless humidifier, or any combination thereof.

The microphone 140 can be used to record sound(s) during a sleep session (e.g., sounds from the user 210) to determine (e.g., using the control system 110) one or more sleep-related parameters, which may include one or more events (e.g. respiratory events), as described in further detail herein. In some implementations, the sound data can be analyzed by the control system 110 to determine a location of an operational issue in the respiratory therapy system and/or one or more causes of an operational issue in the respiratory therapy system. In some such implementations, spectral signatures associated with the sound data may be indicative of the operational issue associated with one or more components of the respiratory therapy system. For example, whistles relating to poor mask fit can be distinguished from whistles due to bad humidifier tub seals or placement by analyzing the sound data.

In some implementations, a second microphone 140 can be coupled to or integrated in the respiratory therapy device 122, the user interface 124, the conduit 126, or the user device 170. In some such implementations, a signal detected by the second microphone (e.g., coupled to or integrated in the respiratory therapy device) could direct the user to download an app to use the first microphone (e.g., coupled to or integrated in an external device) to confirm the possible issue. Additionally or alternatively, based at least in part on a signal detected by the first microphone (e.g., coupled to or integrated in an external device), the app on the external device can be used to activate the second microphone (e.g., coupled to or integrated in the respiratory therapy device) for confirmation.

In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves. In one or more implementations, the sound waves can be audible to a user of the system 100 (e.g., the user 210 of FIG. 2) or inaudible to the user of the system (e.g., ultrasonic sound waves). The speaker 142 can be used, for example, as an alarm clock or to play an alert or message to the user 210 or caregiver (e.g., in response to a movement event and/or change in body position). The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (e.g., a movement, a body position) described in herein, such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 122, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of an operational issue in the respiratory therapy system and/or one or more causes of an operational issue in the respiratory therapy system. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and the RF transmitter 148 are combined as a part of an RF sensor 147 (e.g., a RADAR sensor). In some such implementations, the RF sensor 147 includes a control circuit. The specific format of the RF communication could be Wi-Fi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. For example, the camera 150 may include a thermal imaging camera (or a thermographic camera), such as a Forward Looking Infrared (FLIR) camera. The thermal imaging camera may be used to detect “hot spots” (or other thermal irregularities) on or in the respiratory therapy device 122 or another component of the respiratory therapy system 120, during normal use of the respiratory therapy device 122, during a test of the respiratory therapy device 122, during ramp in speed of the motor, etc.

The image data from the camera 150 can be used by the control system 110 to determine one or more of the sleep-related parameters described herein. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location of the user, to determine chest movement of the user 210, to determine air flow of the mouth and/or nose of the user 210, to determine a time when the user 210 enters the bed 230, and to determine a time when the user 210 exits the bed 230. The camera 150 can also be used to track eye movements, pupil dilation (if one or both of the user 210's eyes are open), blink rate, or any changes during REM sleep. In some implementations, the camera 150 includes a wide angle lens or a fish eye lens.

In some implementations, the camera 150, such as a camera comprised in an external device (e.g., the user device 170, such as a smartphone, of the system 100) relative to the respiratory therapy device 122 (or another component of the respiratory therapy system 120) for locating and/or identifying the operational issue. The camera 150 can be used alone, or in combination with one or more other sensors, to determine a distance such as a distance from the external device to the respiratory therapy device 122 (or another component of the respiratory therapy system 120). For example, the camera 150 may be used in combination with a LiDAR sensor, a sonar sensor, and/or a radar sensor, each of which as described herein, to determine a distance and/or position of the external device relative to the respiratory therapy device 122 (or another component of the respiratory therapy system 120). In some implementations, augmented reality may be incorporated using image data generated by the camera 150 and/or data generated by one or more other sensors such as a LiDAR sensor. For example, augmented reality annotations can be overlaid on an image (e.g., live image) of the respiratory therapy device 122 (or another component of the respiratory therapy system 120) while using the external device. In some implementations, the user may be instructed, via or with the assistance of the augmented reality annotations, to move the external device relative to the respiratory therapy device 122 such as according to a test protocol.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user 210 and/or movement of the user 210. The IR sensor 152 can also be used in conjunction with the camera 150 when measuring the presence, location, and/or causes of the operational issue in the respiratory therapy system 120. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210 (FIG. 2). In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user 210's nose. In still other implementations, the analyte sensor 174 can be positioned near the user 210's mouth when the user interface 124 is a nasal mask or a nasal pillow mask. In some implementations, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be positioned in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example the air inside the user 210's bedroom. The moisture sensor 176 can also be used to track the user 210's biometric response to environmental changes.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not physically and/or communicatively coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Non-physiological parameters can also include operational parameters of the respiratory therapy system, including flow rate, pressure, humidity of the pressurized air, speed of motor, etc. Other types of physiological and non-physiological parameters can also be determined, either from the data from the one or more sensors 130, or from other types of data. Further, the data from the one or more sensors 130 can be analyzed to determine the presence, location, and/or causes of the operational issue in the respiratory therapy system 120.

The user device 170 (FIG. 1) includes a display device 172. The user device 170 can be, for example, a mobile device such as a smart phone, a tablet, a laptop, a gaming console, a smart watch, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for analyzing data associated with a user's use of the respiratory therapy system 120, according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems for implementing the present disclosure can be formed using any portion or portions of the components shown and described herein and/or in combination with one or more other components.

Referring generally to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

In some implementations, the control system 110, the memory device 114, any of the one or more sensors 130, or any combination thereof can be located on and/or in any surface and/or structure that is generally adjacent to the bed 230 and/or the user 210. For example, in some implementations, at least one of the one or more sensors 130 can be located at a first position 255A on and/or in one or more components of the respiratory therapy system 120 adjacent to the bed 230 and/or the user 210. The one or more sensors 130 can be coupled to the respiratory therapy system 120, the user interface 124, the conduit 126, the display device 128, the humidification tank 129, or any combination thereof.

In some implementations, a primary sensor, such as the microphone 140, is configured to generate acoustic data associated with the user 210 during a sleep session. For example, one or more microphones (the same as, or similar to, the microphone 140 of FIG. 1) can be integrated in and/or coupled to (i) a circuit board of the respiratory therapy device 122, (ii) the conduit 126, (iii) a connector between components of the respiratory therapy system 120, (iv) the user interface 124, (v) a headgear (e.g., straps) associated with the user interface, or (vi) any combination thereof.

In some implementations, one or more secondary sensors may be used in addition to the primary sensor to generate additional data. In some such implementations, the one or more secondary sensors include: a microphone (e.g., the microphone 140 of the system 100), a flow rate sensor (e.g., the flow rate sensor 134 of the system 100), a temperature sensor (e.g., the temperature sensor 136 of the system 100), a camera (e.g., the camera 150 of the system 100), a vane sensor (VAF), a hot wire sensor (MAF), a cold wire, a laminar flow sensor, an ultrasonic sensor, an inertial sensor, or any combination thereof.

Additionally, or alternatively, one or more microphones (the same as, or similar to, the microphone 140 of FIG. 1) can be integrated in and/or coupled to a co-located smart device, such as the user device 170, a TV, a watch (e.g., a mechanical watch or the smart device 270), a pendant, the mattress 232, the bed 230, beddings positioned on the bed 230, the pillow, a speaker (e.g., the speaker 142 of FIG. 1), a radio, a tablet, a waterless humidifier, or any combination thereof.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

Generally, the sleep session includes any point in time after the user 210 has laid or sat down in the bed 230 (or another area or object on which they intend to sleep), and has turned on the respiratory therapy device 122 and donned the user interface 124.

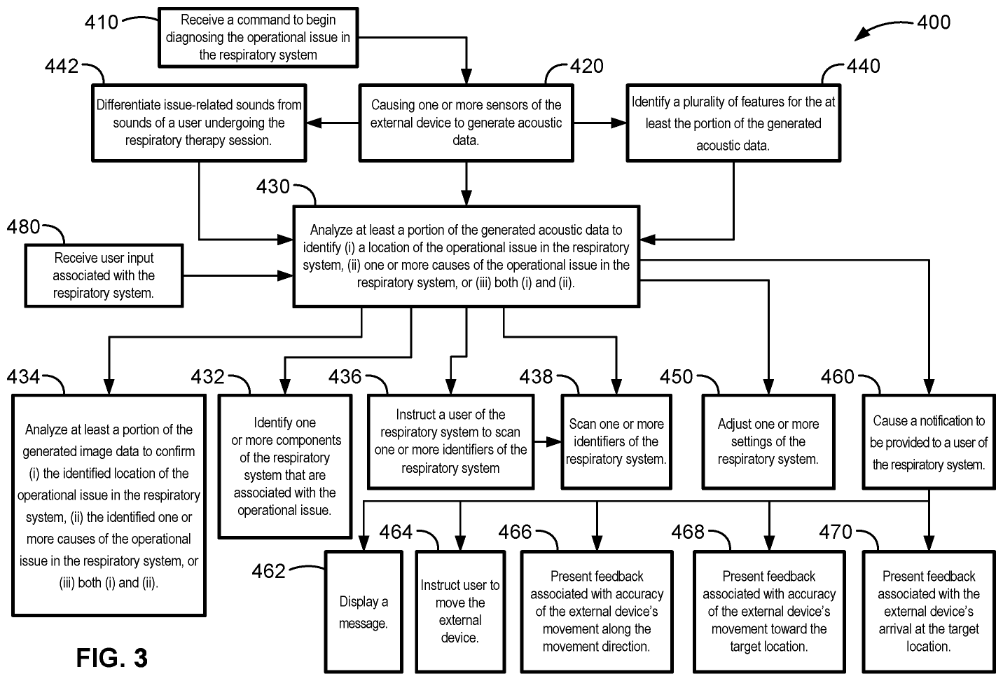

Referring to FIG. 3, a flow diagram for a method 400 for diagnosing an operational issue in a respiratory therapy system is disclosed. One or more steps of the method 400 can be implemented using any element or aspect of the system 100 (FIGS. 1-2) described herein. At step 410 of the method 400, a command is received, via an external device (e.g., the user device 170 of the system 100), to begin diagnosing the operational issue in the respiratory therapy system (e.g., the respiratory therapy system 120). In some implementations, the external device is a mobile phone, a smart speaker, a smart pillow, a smart mattress, a wearable device (e.g., smart glasses, a smart watch), a tablet, a computer, an e-book reader, or a gaming console.

At step 420 of the method 400, one or more sensors (e.g., the microphone 140 and/or the acoustic sensor 141 of the system 100) of the external device are caused to generate acoustic data, which is indicative of one or more sounds emanating from the respiratory therapy system. The one or more sensors may provide directional indication of the sounds emanating from the respiratory therapy system, which may indicate a location of the operational issue. For example, in some implementations, the one or more sensors of the external device include two microphones spaced apart from each other. In some implementations, the one or more sensors of the external device include an array of microphones with beamforming.

In some implementations, the disclosed technology causes the respiratory therapy system to ramp motor speeds, and generate acoustic data at different motor speeds. This allows detection of operational issues that may only manifest at certain motor speeds. For example, there may be irregular sounds (or noises) generated by defective bearings only at certain motor speeds (e.g. resonance speeds). In some implementations, it may be informative to “stress” the motor by intentionally running the respiratory therapy system at high speed, such as 10,000 RPM (aka revolutions per minute). In some implementations, the disclosed technology may be used to generate acoustic data across a single (but full) therapy session, or multiple therapy sessions. This can be useful to detect and/or identify operational issues that might occur sporadically; for example, some operational issues may only occur (i) under certain environmental conditions, such as specific humidifies and/or temperatures, (ii) when any environmental conditions change during a night, and/or (iii) when the respiratory therapy device has been running for a certain duration of time (e.g., a certain number of hours). In some implementations, the external device may be placed near or on (e.g. in a dock) the respiratory therapy device 122, such as for an overnight test. This may usefully allow acoustic data (e.g., via a microphone), and optionally motion data (e.g. via an accelerometer), to be collected over one or more therapy sessions.

In some implementations, the microphone of the external device samples at a rate suitable for detecting sounds associated with operational issues. In some implementations, the microphone of the external device samples at a rate of about 96 kHz, about 48 kHz, or about 24 kHz. Some sounds and/or noises associated with operational issues may be detectable by a microphone, but not audible to certain population of human. Thus, in some aspects, the method 400 provides for early detection of operational issues before the user is able to recognize an unusual noise that is associated with the operational issues.

At step 430 of the method 400, at least a portion of the generated acoustic data is analyzed to identify (i) a location of the operational issue in the respiratory therapy system, (ii) one or more causes of the operational issue in the respiratory therapy system, or (iii) both (i) and (ii). For example, the one or more causes of the operational issue in the respiratory therapy system may be associated with an aged impeller, a blocked filter, a leaky humidifier tub, a leaky gasket, a mask blockage, a power supply defect or wear, a screen defect or wear, failing ball bearing(s), a loose component (such as a screw or other fixing), an improper mounting (such as an incorrectly fitted or loose mounting), a missing or degraded gasket, or any combination thereof. In some implementations, the operational issue is associated with leak of air from the respiratory therapy system. In other implementations, the operational issue is not associated with leak of air from the respiratory therapy system. In further implementations, the operational issue is associated with the respiratory therapy system and detectable when the user is not engaging in respiratory therapy, e.g., is not wearing the user interface. In some implementations, the operational issue is associated with a moving component of the respiratory therapy system when in operation, e.g., the motor, impeller, ball bearing(s), etc.

In some implementations, the analyzing the at least a portion of the generated acoustic data is based, at least in part, on a Cepstrum analysis, an autocepstrum analysis, an auto-correlation analysis, a spectral analysis (e.g., a fast Fourier transform (FFT) optionally with a sliding window, a spectrogram, a short time Fourier transform (STFT), a wavelet-based analysis, or any combination thereof), or any combination thereof. In some implementations, the analysis provides spectral signatures indicative of one or more causes of the operational issues.

In some implementations, the at least a portion of the generated acoustic data may be analyzed in the Fourier domain to diagnose an operational issue in the respiratory therapy system. The analysis of the Fourier sound spectrum may be in any one or more (i) patterns of peaks at integer multiples of the fundamental frequency (harmonics), which is set by the motor speed; (ii) patterns of peaks at non-harmonic frequencies; (iii) “bulges” in predetermined frequency ranges; and/or (iv) correlation between the spectrum (e.g., all or one or more portions of it) and a library of spectrum(s) from healthy motors, unhealthy motors, and/or other known failure modes of the flow generator.

In some implementations, the analyzing the at least a portion of the generated acoustic data may include (i) computing a frequency domain representation of the at least a portion of the generated acoustic data, and (ii) processing the frequency domain representation by, for example, applying an integer-multiple function or a non-integer-multiple function of a fundamental frequency attributable to operation of the motor.

In some implementations, the analyzing the at least a portion of the generated acoustic data may include applying, to the frequency domain representation and to one or more predetermined spectrum(s) from healthy motors, unhealthy motors, and/or other known failure modes of the flow generator, a statistical correlation function. Additionally, or alternatively, in some implementations, the analyzing the at least a portion of the generated acoustic data may include applying to the frequency domain representation a resonant frequency function.

In some implementations, the analyzing the at least a portion of the generated acoustic data can include deriving one or more features from the at least a portion of the generated acoustic data. The one or more features can include (i) position, (ii) frequency, (iii) amplitude, (iv) a noise vector comprising two or more of position, frequency and/or amplitude, or (v) any combination thereof. In some such implementations, the one or more may be classified. For example, the noise vector can be classified to diagnose an operational issue in the respiratory therapy system.

In some implementations, the analyzing the at least a portion of the generated acoustic data may further include computing at least one peak ratio of the peak ratios by dividing a height of a peak by a power (e.g., average power or root mean square (RMS) power) in a band surrounding the peak. The integer-multiple function may extract harmonic peak ratios from peaks in the frequency domain representation at integer multiples of the fundamental frequency. The noise vector may be derived, for the classifying, with output data from the applying of the integer-multiple function.

The non-integer-multiple function may extract non-harmonic peak ratios from peaks in the frequency domain representation at non-integer multiples of the fundamental frequency. The noise vector may be derived, for the classifying, with output data from the applying of the non-integer-multiple function. The analyzing may include applying, to the frequency domain representation, a resonant frequency function, and may further include deriving the noise vector with an output data set from the resonant frequency function. The output data set from the resonant frequency function may include power data for one or more predetermined resonant regions. The power data may include a noise ratio. The noise ratio may include an average power of a predetermined resonant region divided by a power of a reference region.

Applying the non-integer-multiple function may further include thresholding peak ratios using one or more first predetermined ratio thresholds, such as a predetermined ratio threshold. The classifying may include counting harmonic peak ratios in the noise vector that exceed one or more second predetermined thresholds, such as a predetermined threshold. The classifying may include forming a weighted sum from harmonic peak ratios generated with the integer-multiple function.

In some implementations, the analyzing the at least a portion of the generated acoustic data may include accessing one or more (e.g., a library of) spectrums from healthy motors, unhealthy motors, and/or other known failure modes of the flow generator. In some such implementations, the analyzing the at least a portion of the generated acoustic data may include applying, to the frequency domain representation and the one or more spectrums from healthy motors, unhealthy motors, and/or other known failure modes of the flow generator, a statistical correlation function. For example, the noise vector may be derived, for the classifying, with output data from the applying of the statistical correlation function. In some such examples, the classifying the noise vector includes comparing the output data from the applying of the statistical correlation function to one or more correlation thresholds.

In some implementations, the analyzing the at least a portion of the generated acoustic data may include parsing, for the computing of the frequency domain representation, the acoustic signal into a plurality of intervals that each coincide with a period of time of a sub-portion of a respiratory event type, which may be taken from a plurality of consecutive or non-consecutive respiratory events. The respiratory event type may include a detected apnea and/or a detected breath cycle. The period of time of the sub-portion of the respiratory event includes a detected interval coinciding with a predetermined blower motor speed that is approximately constant. In some such implementations, the method may include combining, such as by the processor or controller, the parsed intervals for the computing of the frequency domain representation. In some such implementations, the method may include determining, such as by the processor or controller, that the combined parsed intervals at least satisfy a minimum quantity. The minimum quantity may include a breath count associated with the combined parsed intervals or an accumulated time amount of the combined parsed intervals. The classifying may evaluate a weighed sum of integer multiple peak ratios, non-integer multiple peak ratios, and noise ratios. The classifying may evaluate each of a weighed sum of integer multiple peak ratios, a weighted sum of non-integer multiple peak ratios, and a weighted sum of noise ratios.

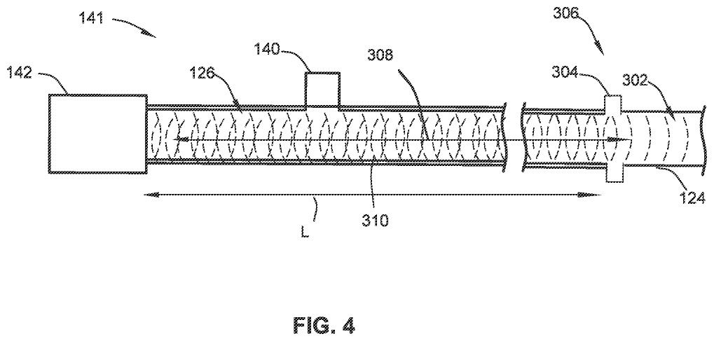

Referring to FIG. 4, an example method of generation (step 420) and analysis (step 430) of the acoustic data is illustrated, according to aspects of the present disclosure. Specifically, FIG. 4 illustrates the generation of acoustic data (step 420 of the method 400), in the form of echo data, in response to an acoustic reflection indicative of one or more features of a user interface (e.g., the user interface 124). In some implementations, the generated acoustic data can be analyzed to determine presence, location, and/or causes of one or more operational issues associated with the user interface. Spectral signatures associated with the acoustic data may be indicative of the one or more operational issues associated with the user interface.

The generation of the acoustic data can involve the acoustic sensor 141, which includes the microphone 140 (or any type of audio transducer) and a speaker 142. However, as discussed above, the speaker 142 can instead be replaced by another device that can generate an acoustic signal, such as the motor of the respiratory therapy device 122. In some implementations, the acoustic signal may be a vibratory signal detectable by a transducer (e.g., the microphone 140) or by another sensor (e.g., the motion sensor 138 such as a gyroscope and/or an accelerometer). In some implementations, vibrations detected by the motion sensor 138 is used to complement (e.g., to confirm or supplement) the analyses performed using the acoustic data. Alternatively, in some implementations, vibrations detected by the motion sensor 138 is used in place of the acoustic data generated by the acoustic sensor 141. The microphone 140 and speaker 142 are shown in specific locations relative to the conduit 126, which is connected to the respiratory therapy device 122 (not shown). However, the locations of the microphone 140 and the speaker 142 can vary from what is shown, as discussed above. Examples of generation and analysis of echo data are described in International Publication No. WO 2010/091462, which is hereby incorporated by reference herein in its entirety.

The speaker 142 emits an acoustic signal 302 within the conduit 126. The acoustic signal 302 is in the form of a sound. The sound can be one or more of a standard sound (e.g., an original, unmodified sound from an off-the-shelf sound application), a custom sound, an inaudible frequency, a white noise sound, a broad band impulse, a continuous sinusoidal waveform, a square waveform, a sawtooth waveform, and a frequency modulated sinusoid (e.g., chirp). According to some other implementations, the acoustic signal 302 can be in the form of one or more of an audible sound or an ultrasonic sound. According to some other implementations, the acoustic signal 302 is in the form of an inaudible sound, where the sound is inaudible based on one or both of the frequency of the sound (e.g., the frequency being outside of the frequency range for human hearing) or the amplitude of the sound (e.g., the amplitude being low enough that the sound is not loud enough for human perception).

In one or more implementations, the acoustic signal 302 is emitted at specific times, such as when the user first puts on the user interface, after the user takes off the user interface, after detecting that an apnea event or a hypopnea event is occurring (e.g., after detecting using a respiratory therapy device that an apnea event or a hypopnea event is occurring). For example, the specific monitoring times are selected to be at intervals of 0.1 seconds for a duration of at least 4 seconds. In some such implementations, the puffs of air emitted during “autostart” (i.e., when respiratory system emits puffs of air to detect whether the user is wearing the user interface) can be used to generate the acoustic signal.

The acoustic signal 302 travels down the length L of the conduit 126 until the acoustic signal 302 contacts a feature 304 of the user interface 124 at or near a connection 306 of the user interface 124 with the conduit 126. The feature 304 includes a widening of the pathway 308 formed through the conduit 126 and the user interface 124. The widening at the feature 304 causes a change in the acoustic impedance of the acoustic signal 302 and an acoustic reflection 310. The acoustic reflection 310 travels back down the length L of the conduit 126 until it reaches the microphone 140. The microphone 140 detects the acoustic reflection 310 and generates acoustic data (i.e. echo data) in response to the acoustic reflection 310. The generated data can be subsequently analyzed for determining the presence, location, and/or one or more causes of an operational issue associated with the user interface 124.

The acoustic signal 302 can continue beyond the feature 304 and into the user interface 124. Although not illustrated, the user interface 124 can include one or more additional features that change the acoustic impedance of the acoustic signal 302, which further generates the acoustic reflection 310. Thus, although only the feature 304 is illustrated and described as causing the acoustic reflection 310, there can be multiple features of the user interface 124 that can all contribute to the acoustic reflection 310.

As such, in some implementations, the system 100 is configured to generate acoustic data, which is in turn analyzed by the control system 110. In some implementations, the acoustic data includes reflected sound waves received by a microphone (e.g., the microphone 140 of the system 100) that are transmitted from a speaker (e.g., the speaker 142 of the system 100, or an external speaker). The reflected sound waves are indicative of shapes and dimensions of the components in the sound waves' path(s). Additionally or alternatively, the acoustic data includes sound(s) from the user that is indicative of one or more sleep-related parameters (e.g., breathing through the nose, breathing through the mouth, snoring, sniffling).

For example, the acoustic data can include data generated by the microphone 140. The speaker 142 generates a sound. The sound can travel through the humidification tank 129, along a first connection, along the conduit 126, via a second connection, via a waterless humidifier (if fitted), to one or more mask cavities (e.g., nostrils and/or mouth), to the user's respiratory system (including nose and/or mouth, airway(s), lungs, etc.). For each change or feature in the path (e.g., a cavity, a junction, a change in shape and/or in length of the conduit which may be caused by movement of the conduit and/or another component of the respiratory therapy system), a reflection at that point is seen, the location of which may be determined based on the known speed of sound. The generated acoustic data can then be analyzed (e.g., at step 430 of the method 400) to diagnose operational issues of the respiratory therapy system, using one or more methods disclosed herein.

In some implementations, a cepstrum analysis is implemented to analyze the acoustic data (step 430 of the method 400). Cepstrum is a “quefrency” domain, which is also known as the spectrum of the log of a time domain waveform. For example, a cepstrum may be considered the inverse Fourier Transform of the log spectrum of the forward Fourier Transform of the decibel spectrum, etc. The operation essentially can convert a convolution of an impulse response function (IRF) and a sound source into an addition operation so that the sound source may then be more easily accounted for or removed so as to isolate data of the IRF for analysis. Techniques of cepstrum analysis are described in detail in a scientific paper entitled “The Cepstrum: A Guide to Processing” (Childers et al, Proceedings of the IEEE, Vol. 65, No. 10, October 1977) and Randall R B, Frequency Analysis, Copenhagen: Bruel & Kjaer, p. 344 (1977, revised ed. 1987).

Such a method may be understood in terms of the property of convolution. The convolution off and g can be written as f*g. This operation can be the integral of the product of the two functions (f and g) after one is reversed and shifted. As such, it is a type of integral transform as Equation 1, as follows:

(f*g)(t)=∫_{-∞}^{∞}f(τ)·g(t−τ)dτ Eq. 1

While the symbol t is used above, it need not represent the time domain. But, in that context, the convolution formula can be described as a weighted average of the function f(τ) at the moment t where the weighting is given by g(−τ) simply shifted by amount t. As t changes, the weighting function emphasizes different parts of the input function.

More generally, if f and g are complex-valued functions on R^{d}, then their convolution may be defined as the integral of Equation 2:

(f*g)(x)=∫_{R}_{_{d}}f(y)g(x−y)dy=∫_{R}_{_{d}}f(x−y)g(y)dy Eq. 2

A mathematical model that can relate an acoustic system output to the input for a time-invariant linear system, such as one involving conduits of a respiratory treatment apparatus, (which may include some human or other unknown part of the system) can be based on this convolution. The output measured at a microphone of the system may be considered as the input noise “convolved” with the system Impulse Response Function (IRF) as a function of time (t), as shown in Equation 3:

y(t)=s₁(t)*h₁(t) Eq. 3

where * denotes the convolution function; y(t) is the signal measured at the sound sensor; S₁(t) is the sound or noise source such, as a noise or sound created in or by a flow generator of a respiratory treatment apparatus; and h₁(t) is the system IRF from the noise or sound source to the sound sensor. The Impulse Response Function (IRF) is the system response to a unit impulse input.

Conversion of Equation 3 into the frequency domain by means of the Fourier Transform of the measured sound data (e.g., a discrete Fourier Transform (“DFT”) or a fast Fourier transform (“FFT”) and considering the Convolution Theorem, Equation 4 is produced:

$$y ( t ) = {s}_{1} ( t ) * {h}_{1} ( t ) {\to }^{Fourier Transform} Y ( f ) = {S}_{1} ( f ) {H}_{1} ( f ) \qquad Eq . 4$$

where Y(f) is the Fourier Transform of y(t); S₁(f) is the Fourier Transform of s₁(t); and H₁(f) is the Fourier Transform of h₁(t). In such a case, convolution in the time domain becomes a multiplication in the frequency domain.

A logarithm of Equation 4 may be applied so that the multiplication is converted into an addition, resulting in Equation 5:

Log{Y(f)}=Log{S₁(f)H₁(f)}=Log{S₁(f)}+Log{H₁(f)} Eq. 5

Equation 5 may then be converted back into the time domain, by an Inverse Fourier Transform (IFT) (e.g., an inverse DFT or inverse FFT), which results in a complex cepstrum (K(τ)) (complex because one can work from the complex spectrum)—the inverse Fourier Transform of the logarithm of the spectrum; Equation 6.

K(τ)=IFT[Log{S₁(f)}+Log{H₁(f)}] Eq. 6

where “τ” is a real valued variable known as quefrency, with units measured in seconds. From this, the effects that are convolutive in the time domain become additive in the logarithm of the spectrum, and remain so in the cepstrum.

Consideration of the data from a cepstrum analysis, such as examining the data values of the quefrency, may provide information about the system. For example, by comparing cepstrum data of a system from a prior or known baseline of cepstrum data for the system, the comparison, such as a difference, can be used to recognize differences or similarities in the system that may then be used to implement varying functions or purposes disclosed herein. The following disclosure can utilize the methodologies of such an analysis, as herein explained, to implement the determination of any operational issue associated with the respiratory therapy system.

In some implementations, direct spectral methods can be implemented to analyze the acoustic data. Some examples of direct spectral methods include processing discrete Fourier transform (DFT), fast Fourier transform (FFT) optionally with a sliding window, short time Fourier transform (STFT), wavelets, wavelet-based cepstrum calculation, deep neural networks (e.g., using imaging methods applied to spectrograms), Hilbert-Huang transform (HHT), empirical mode decomposition (EMD), blind source separation (BSS), Kalman filters, or any combination thereof. In some implementations, cepstral coefficients (CCs) such as mel-frequency cepstral coefficients (MFCCs) may be used, for example, by treating the acoustic data analysis in a similar manner as speech recognition and using a machine learning/classification system.

Referring back to FIG. 3, in some implementations, the acoustic data generated by the microphone (and the accelerometer optionally) may be analyzed together with data from the respiratory therapy system 120, such as the motor blower (e.g., generating thermal readings from the respiratory therapy device 122). A change in acoustic spectrum, combined with a small increase in current consumption and/or an elevated operating temperature versus ambient temperature can be then used to detect subtle degradation and/or impending failure of the motor. For example, a frequency analysis can be performed by comparing the spectrum of the motor associated with the current respiratory therapy system with the spectrum of healthy motors, unhealthy motors, and/or other known failure modes of the flow generator. Suspect but unknown failure modes may also be captured and/or uploaded to the cloud for further analysis by a deep neural network.

In some implementations, for example, where the external device 170 is placed against the respiratory therapy device 122 and/or on top of the respiratory therapy device 122 and/or directly adjacent to the flow generator, the microphone of the external device 170 and the accelerometer of the external device 170 may both be used to analyze the vibration through the housing of the respiratory therapy device 122. For example, the motion sensor 138 may be an accelerometer comprised in a smart device (e.g., a smartphone), which can be used to detect aberrant vibrations from the respiratory therapy device 122 by being placed on or near the respiratory therapy device 122 (e.g., near the motor, when motor is running, at one or more speeds, and/or during a ramp in speed of the motor).

In some implementations, a sound level is measured before, during, and optionally after the respiratory therapy device 122 runs through one or more test modes, and/or the respiratory therapy device 122 runs through a normal operating mode. Prior acoustic data associated with one or more components (e.g., the blower) of the respiratory therapy system 120 may then be analyzed to understand the ambient stationary and nonstationary noise in the environment, so as not to be confused and/or confounded by other noises. Additionally or alternatively, in some implementations, speech or other sound detection and/or notification can be utilized to (i) request the user to keep the environment quiet, so that the tests can be carried out, and/or (ii) exclude periods of a recording that might have interference (e.g., speech, background noise, pops, bangs). In some implementations, de-noising of the acoustic data may be performed. For example, a digital filter, such as a Kalman filter (also known as linear quadratic estimation), may be used to filter (e.g., normalize or remove) statistical noise and/or other inaccuracies. In some such implementations, the de-noising includes estimating a joint probability distribution over the variables in the acoustic data for each timeframe.

The analysis of the generated acoustic data can be advantageous to characterize, such as based on resonances, the types of sounds and/noises associated with particular operational issues in the respiratory therapy system. For example, when bearings wear out, they produce non-linear resonances, and not a single tone. In some implementations, the Cepstrum analysis can be used to diagnose motor and/or bearing issues. In addition, low-frequency ultrasonic sounds (e.g., 20-24 kHz) may be picked up by the external device, while being inaudible to human.

In some implementations, one or more components of the respiratory therapy system that are associated with the operational issue are identified at step 432. For example, the one or more components of the respiratory therapy system can include the respiratory therapy device 122, the user interface 124, the conduit 126, the display device 128, the humidifier tank 129, one or more sensors 130 of the respiratory therapy system 120, or any combination thereof. In some implementations, the location of the operational issue in the respiratory therapy system is associated with the one or more causes of the operational issue in the respiratory therapy system. In some implementations, the determined location is associated with one or more components of the respiratory therapy system.

In some implementations, a user may be instructed to remove and/or replace a specific component within the respiratory therapy system that is identified as the likely cause and/or location of the operational issue. For example, if the humidifier tank 129 is the suspected cause and/or location of the operational issue, step 432 of the disclosed method 400 may further include instructing a user to (i) remove, replace and/or clean the humidifier tank, (ii) remove, replace, and/or clean one or more seals between the humidifier tank and the respiratory therapy device, and/or (iii) remove, replace, and/or clean one or more seals between the humidifier tank and the air pathway. In some implementations, when one or more components of the respiratory therapy system that are associated with the operational issue are identified at step 432, the method 400 further includes carrying out a test to confirm the identity of the one or more components, and detecting acoustic data during the test, optionally ramping the motor speed to help detect issues that might only occur at certain motor speeds.

In some implementations, one or more sensors of the external device further includes a camera (e.g., the camera 150 of the system 100) configured to generate image data reproducible as one or more images. In some such implementations, at step 434, at least a portion of the generated image data is analyzed to confirm (i) the identified location of the operational issue in the respiratory therapy system, (ii) the identified one or more causes of the operational issue in the respiratory therapy system, or (iii) both (i) and (ii). In some implementations, the external device may be configured to provide directional instructions to the user to move the camera closer and/or allow further image data capture to confirm the location and/or causes of the operational issue in the respiratory therapy system, such as what is disclosed in steps 462, 464, 466, 468, and 470 herein. Additionally or alternatively to the camera, in some implementations, UWB or LiDAR sensors of the external device may be used to better measure distance between the location of the operational issue and the external device.

For example, the external device may be configured to capture real-time video data. When a shape of the respiratory therapy device 122 and/or a shape of the user interface 124 is detected, an overlay of directional instructions and/or marks can be placed on a display device (e.g., the display device 172) of the external device to instruct the user to move the external device closer to the respiratory therapy device 122 or the user interface 124. The overlay may include directional arrows that point the user to the correct location to better capture the acoustic data (step 420) and/or the image data.

Additionally or alternatively, in some implementations, at step 436, a user of the respiratory therapy system is instructed to scan one or more identifiers of the respiratory therapy system. Further additionally or alternatively, in some implementations, at step 438, via the camera of the external device, the one or more identifiers of the respiratory therapy system is scanned (e.g., automatically or manually). For example, the one or more identifiers may include a serial number, a QR code, or both, which can be used to identify one or more components of the respiratory therapy system (e.g., the respiratory therapy device, the user interface, the conduit, the display device, the humidifier, one or more sensors of the respiratory therapy system, or any combination thereof).

In some implementations, at step 440, a plurality of features is identified for the at least the portion of the generated acoustic data. For example, in some implementations, the plurality of features can include (i) a spectral signature or a change in spectral signature (e.g., relative to a correctly operating component), (ii) a frequency or a change in frequency, (iii) an amplitude or a change in amplitude, or (iv) any combination thereof.

In some implementations, at step 420, the one or more sensors of the external device are configured to generate acoustic data over a respiratory therapy session. In some such implementations, at step 442, issue-related sounds are differentiated from sounds of a user undergoing the respiratory therapy session. Examples of sounds of the user undergoing the respiratory therapy session include: breathing, coughing, sneezing, crying, mumbling, talking, spitting, swallowing, snorting, etc. In some such implementations, acoustic data associated with a user of the respiratory therapy system may be received from an internal microphone of the respiratory therapy system during a respiratory therapy session. The acoustic data generated by the one or more sensors of the external device is differentiated from the acoustic data received from the internal microphone of the respiratory therapy system, to exclude sounds of a user undergoing the respiratory therapy session (e.g., sounds associated with the user sleeping, such as snoring, breathing, coughing, or any combination thereof).

In some implementations, the disclosed technology may employ noise cancellation to mitigate user and/or environmental noise. For example, a transfer learning neural network may be used to (i) recognize user-related and/or environment-related noises and/or (ii) identify those that should be removed from the acoustic data. Additionally, or alternatively, in some implementations, the user can be instructed that such user-related and/or environment-related noise is being detected, and should be reduced or stopped (e.g. turn off TV, stop talking, etc.).

Additionally, or alternatively, in some implementations, the internal microphone, alone or in combination with another sensor (e.g., an external microphone or a different sensor), is capable generating acoustic data for the detection and/or identification of the operational issues disclosed herein. The analysis of the acoustic data generated by the internal microphone can be carried out in the same, or similar, manner as that disclosed herein regarding analyzing the acoustic data generated by the external microphone (e.g., in an external device 170).

For example, in some implementations, the internal microphone is used to detect operational issues associated with the respiratory therapy device (or another component of the respiratory therapy system) by detecting acoustic signals in the airflow generated by the respiratory therapy device 122. The internal microphone may be in, or in a fluid communication with, an air flow path of the motor/pressure generator and/or the air circuit. As another example, in some implementations, when the internal microphone is used in combination with an external microphone, the internal microphone is configured to detect acoustic signals in the air circuit, whereas the external microphone is configured to detect acoustic signals emanating from respiratory therapy device. In some such implementations, in response to detecting an operational issue using the acoustic data generated by the external microphone, such determination may be verified by analyzing the acoustic data generated by the internal microphone simultaneously with or subsequently to the external microphone's detection of the operational issue. In some other implementations, in response to detecting an operational issue using the acoustic data generated by the internal microphone, such determination may be verified by analyzing the acoustic data generated by the external microphone simultaneously with or subsequently to the internal microphone's detection of the operational issue. As yet a further example, in some implementations, in response to detecting an operational issue using the acoustic data generated by the internal microphone or the external microphone, instruction can be sent to the user to carry out a test using the same or a different microphone to confirm any detection of a potential operational issue.

In some implementations, at step 450, responsive to the identified one or more causes of the operational issue in the respiratory therapy system (step 430), one or more settings of the respiratory therapy system are adjusted, such as (i) preventing the respiratory therapy system from turning on, (ii) preventing one or more components of the respiratory therapy system from turning on, or (iii) both (i) and (ii).

In some implementations, at step 460, responsive to the analyzing the at least the portion of the generated acoustic data, a notification is caused to be provided (e.g., via the external device) to a user of the respiratory therapy system. In some such implementations, at step 462, a message is displayed on the external device. Additionally or alternatively, in some such implementations, at step 464, the user is instructed (e.g., via the external device) to move the external device. As disclosed herein, in some implementations, using the external device allows the external device to be moved around the respiratory therapy system without disturbing the respiratory therapy system. Further, the external device may be switched and/or upgraded independently from the respiratory therapy system.

For example, in some implementations, the notification may be indicative of a movement direction or target location for moving the external device relative to one or more components of the respiratory therapy system. In some such implementations, at step 466, feedback associated with accuracy of the external device's movement along the movement direction is provided to the user. Additionally or alternatively, in some such implementations, at step 468, feedback associated with accuracy of the external device's movement toward the target location is provided to the user. Additionally or alternatively, in some such implementations, at step 470, feedback associated with the external device's arrival at the target location is provided to the user.

In some implementations, at step 480, user input associated with the respiratory therapy system may be received via the external device, such that (i) the location of the operational issue in the respiratory therapy system, (ii) the one or more causes of the operational issue in the respiratory therapy system, or (iii) both (i) and (ii) are identified (step 430) based at least in part on the received user input. For example, the user input can include a photo associated with the operational issue, a video associated with the operational issue, a self-reported complaint of noise and type of noise (e.g., when the noise was heard, for how long the noise was heard, the annoyance level).

Generally, the method 400 can be implemented using a system having a control system with one or more processors, and a memory storing machine readable instructions. The controls system can be coupled to the memory; the method 400 can be implemented when the machine readable instructions are executed by at least one of the processors of the control system. The method 400 can also be implemented using a computer program product (such as a non-transitory computer readable medium) comprising instructions that when executed by a computer, cause the computer to carry out the steps of the method 400.

While the system 100 and the method 400 have been described herein with reference to a single user, more generally, the system 100 and the method 400 can be used with a plurality of users simultaneously (e.g., two users, five users, 10 users, 20 users, etc.). For example, the system 100 and the method 400 can be used in a cloud monitoring setting.

Additionally, or alternatively, in some implementations, the system 100 and/or the methods 600, 800, 1000 can be used to monitor one or more patients while using one or more respiratory therapy systems (e.g., a respiratory therapy system described herein). For example, in some such implementations, a notification associated with the one or more operational issues can be sent to a monitoring device or personnel. The presence, location, and/or cause of the one or more operational issues can be determined using one or more steps of the method 400. Additionally, or alternatively, in some implementations, the presence, location, and/or cause of the one or more operational issues are simply being recorded.

One or more elements or aspects or steps, or any portion(s) thereof, from one or more of any of claims 1-49 below can be combined with one or more elements or aspects or steps, or any portion(s) thereof, from one or more of any of the other claims 1-49 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method for diagnosing an operational issue in a respiratory therapy system, comprising:
receiving, via an external device, a command to begin diagnosing the operational issue in the respiratory therapy system;
causing one or more sensors of the external device to generate acoustic data and image data, the acoustic data being indicative of one or more sounds emanating from the respiratory therapy system, and the image data being reproducible as one or more images; and
analyzing at least a portion of the generated acoustic data and the image data to identify (i) a location of the operational issue in the respiratory therapy system, (ii) one or more causes of the operational issue in the respiratory therapy system, or (iii) both (i) and (ii).

2. (canceled)

3. (canceled)

4. The method of claim 1, wherein the analyzing the at least the portion of the generated acoustic data includes identifying one or more components of the respiratory therapy system that are associated with the operational issue.

5. (canceled)

6. (canceled)

7. The method of claim 1, wherein the one or more sensors of the external device further include a microphone configured to generate or detect at least a portion of the acoustic data, (ii) a speaker configured to generate or emit sound waves, where the microphone is further configured to detect reflections of the generated or emitted sound waves from the speaker, or (iii) a combination thereof.

8. (canceled)

9. (canceled)

10. The method of claim 1, further comprising:
analyzing at least a portion of the generated image data to confirm (i) the identified location of the operational issue in the respiratory therapy system, (ii) the identified one or more causes of the operational issue in the respiratory therapy system, or (iii) both (i) and (ii).

11. (canceled)

12. The method of claim 1, further comprising:
scanning, via a camera of the external device, one or more identifiers of the respiratory therapy system.

13. (canceled)

14. (canceled)

15. The method of claim 12, wherein the one or more identifiers are associated with one or more components of the respiratory therapy system, and wherein the one or more components of the respiratory therapy system includes a respiratory therapy device, a user interface, a conduit, a display device, a humidifier, one or more sensors of the respiratory therapy system, or any combination thereof.

16-20. (canceled)

21. The method of claim 1, wherein the analyzing the at least the portion of the generated acoustic data is based, at least in part, on a fast Fourier transform (FFT) analysis, a short time Fourier transform (STFT) analysis, a spectral analysis, a Cepstrum analysis, an autocepstrum analysis, an auto-correlation analysis, or any combination thereof.

22. (canceled)

23. The method of claim 1, wherein the analyzing the at least the portion of the generated acoustic data includes identifying a plurality of features for the at least the portion of the generated acoustic data.

24. (canceled)

25. The method of claim 1, further comprising:
responsive to the identified one or more causes of the operational issue in the respiratory therapy system, adjusting one or more settings of the respiratory therapy system.

26. The method of claim 25, wherein the adjusting one or more settings of the respiratory therapy system includes (i) preventing the respiratory therapy system from turning on, (ii) preventing one or more components of the respiratory therapy system from turning on, or (iii) both (i) and (ii).

27. The method of claim 1, further comprising:
responsive to the analyzing the at least the portion of the generated acoustic data, causing a notification to be provided, via the external device, to a user of the respiratory therapy system.

28. (canceled)

29. (canceled)

30. The method of claim 27, wherein the providing the notification is indicative of a movement direction or target location for moving the external device relative to one or more components of the respiratory therapy system.

31. The method of claim 30, wherein the providing the notification includes presenting feedback associated with accuracy of the external device's movement along the movement direction.

32-36. (canceled)

37. The method of claim 1, further comprising:
receiving, from an internal microphone of the respiratory therapy system, acoustic data associated with a user of the respiratory therapy system during a respiratory therapy session; and
differentiating the acoustic data generated by the one or more sensors of the external device from the acoustic data received from the internal microphone of the respiratory therapy system to exclude sounds of a user undergoing the respiratory therapy session.

38. (canceled)

39. (canceled)

40. The method of claim 1, further comprising:
receiving, from an internal microphone of the respiratory therapy system, internal acoustic data associated with a user of the respiratory therapy system.

41. The method of claim 40, further comprising:
analyzing at least a portion of the received internal acoustic data to confirm (i) the identified location of the operational issue in the respiratory therapy system, (ii) the identified one or more causes of the operational issue in the respiratory therapy system, or (iii) both (i) and (ii).

42. The method of claim 40, wherein the internal acoustic data associated with the user of the respiratory therapy system is received from the internal microphone in response to analyzing the acoustic data generated by the one or more sensors of the external device, and the command to begin diagnosing the operational issue in the respiratory therapy system is generated in response to analyzing at least a portion of the received internal acoustic data.

43. (canceled)

44. The method of claim 1, further comprising receiving, via the external device, user input associated with the respiratory therapy system, wherein (i) the location of the operational issue in the respiratory therapy system, (ii) the one or more causes of the operational issue in the respiratory therapy system, or (iii) both (i) and (ii) are identified based at least in part on the received user input.

45. (canceled)

46. A system comprising:
a control system including one or more processors; and
a memory having stored thereon machine readable instructions;
wherein the control system is coupled to the memory, and the one or more processors are configured to execute the machine readable instructions to:
receive, via an external device, a command to begin diagnosing an operational issue in a respiratory therapy system;
cause one or more sensors of the external device to generate acoustic data and image data, the acoustic data being indicative of one or more sounds emanating from the respiratory therapy system, and the image data being reproducible as one or more images; and
analyze at least a portion of the generated acoustic data and the image data to identify (i) a location of the operational issue in the respiratory therapy system, (ii) one or more causes of the operational issue in the respiratory therapy system, or (iii) both (i) and (ii).

47. (canceled)

48. A non-transitory computer readable storage medium storing machine readable instructions that, when executed by one or more processors of a control system, cause the one or more processors to:
receive, via an external device, a command to begin diagnosing an operational issue in a respiratory therapy system;
cause one or more sensors of the external device to generate acoustic data and image data, the acoustic data being indicative of one or more sounds emanating from the respiratory therapy system, and the image data being reproducible as one or more images; and
analyze at least a portion of the generated acoustic data and the image data to identify (i) a location of the operational issue in the respiratory therapy system, (ii) one or more causes of the operational issue in the respiratory therapy system, or (iii) both (i) and (ii).

49. (canceled)
